(19) United States
(12) Patent Application Publication
(10) Pub. No.: US 2023/0271991 A1
(43) Pub. Date: Aug. 31, 2023

(54) ALKALINE HYDROLYSIS OF WASTE CELLULOSE

(71) Applicant: NOVAMONT S.P.A., Novara (IT)

(72) Inventors: Catia BASTIOLI, Novara (IT); Luigi CAPUZZI, Novara (IT); Marco COTTI COMETTINI, Brusnengo (BI) (IT); Davide PERINI, Romentino (NO) (IT); Mattia TORCHIO, Quarona (VC) (IT)

(21) Appl. No.: 18/004,684

(22) PCT Filed: Jul. 8, 2021
(86) PCT No.: PCT/EP2021/069078

§ 371 Date: Jan. 9, 2023

(30) Foreign Application Priority Data
Jul. 9, 2020 (IT) 102020000016651

(51) Int. Cl. C07H 1/08; C07H 3/06; C12P 19/04

(52) U.S. Cl. CPC C07H 1/08; C07H 3/06; C12P 19/04; C12P 2201/00

(57) ABSTRACT

The present invention relates to a process which makes it possible to obtain a plurality of organic compounds that can be used as chemical intermediates through the use of waste cellulosic biomass as a raw material. Through this process fermentable saccharides can be extracted, separated and recovered from said waste cellulosic biomass.

Part of the activities that led to the invention were carried out within the project funded by the Bio Based Industries Joint Undertaking Public-Private Partnership under the European Union’s Horizon 2020 research and innovation programme, under Grant Agreement No. 745746.

The present invention relates to a process through which a plurality of organic compounds that can be used as chemical intermediates may be obtained from the use of waste cellulosic biomass as a raw material. By means of this process fermentable saccharides can be extracted, separated and recovered from said waste cellulosic biomass.

Waste cellulosic biomass according to the present invention may be derived from hygiene products, such as disposable baby nappies, adult incontinence pads, feminine hygiene products, cot liners, absorbent materials for general hygiene and personal care, and toilet paper. Such biomass may be post-industrial and/or post-consumer, and in the latter case comes from the sorting of waste or sewage treatment plants.

The hygiene products listed above typically comprise a cellulosic fraction (e.g. cellulose fibres obtained from different plant biomasses, for example through the Kraft process) and may include super-absorbent polymers and an outer covering, usually consisting of non-woven fabric or plastics film. Although these products are commonly sent to landfill or incinerated after use, in recent years processes have been developed to recover and recycle their constituent materials. In order to be able to reuse these products, a process to separate their main components - plastics, Absorbent Hygiene Products waste cellulose (AHPwc) and Super Absorbent Polymer (SAP) - is required. For example, the FaterSMART company has developed a process for separating these components, described in patent application WO 2017/015242. This process includes a stage of treating the personal absorbent products using an autoclave and a dryer to sterilise, pre-separate and dry the materials, eliminating unpleasant odours and potential pathogens, followed by separation and recovery of the cellulosic fraction, the plastics and the super-absorbent polymer.

Sewage treatment plants treat waste water of urban or industrial origin. This wastewater may contain cellulosic biomass, components that become waste to be burned or sent to landfill. These components, if effectively separated from the wastewater and treated, can be reused in other processes, e.g. as a renewable source of fermentable sugars. The KNN Cellulose company has for example developed the ‘Cellvation®’ process for recovering the cellulosic fraction from wastewater treatment sludge. The resulting product, Recell®, is a cellulose suitable for use in the production of sustainable coatings and chemicals in various sectors such as construction and the paper and board industry.

Cellulosic biomass typically comprises a cellulosic fraction rich in polysaccharides (for example hemicellulose and cellulose) consisting of saccharide units with 5 and 6 carbon atoms (referred to as C5-C6 sugars), which is an important renewable source of fermentable sugars. However, because of the complex structure of the cellulosic fraction, the chemical bonds between its structural components (cellulose, hemicellulose and lignin) have to be broken to facilitate enzyme hydrolysis of the polysaccharides into simple sugars. Pre-treatments are therefore commonly used to destroy the outer structure of lignin and hemicellulose, reduce the crystallinity and degree of polymerisation of the cellulose, and allow hydrolytic enzymes access to the cellulose.

Such pre-treatment may be physical, chemical and/or biological in nature.

Patent EP 2 828 392 describes a process for the production of sugars from oleaginous herbaceous plants, comprising alkaline pre-treatment of the lignocellulosic biomass to remove lignin, acetates, extractables and ash, and to allow hemicellulose and cellulose to be recovered, avoiding the formation of degradation by-products such as furfural, HMF and its derivatives.

Patent application US 2010/0112242 describes a method for using biomass of plant and animal origin and municipal waste to produce biofuels. Such biomass undergoes a treatment selected from radiation, sonication, pyrolysis and oxidation in order to modify its molecular structure and obtain sugars.

Patent application WO 2017/015242 describes a method for de-structuring a post-consumer cellulosic biomass by treatment with high temperature and high pressure. After treatment the cellulosic fraction is directly saccharified, producing sugars used as a carbon source by microorganisms for biofuel production.

However, obtaining fermentable C5-C6 sugars from waste cellulosic biomass is difficult, not only because of the complex structure of cellulose, but also because of the presence of impurities, such as the super-absorbent polymer, when not completely separated from the cellulosic fraction, and/or any organic and/or inorganic residues linked to utilisation of the cellulosic biomass itself.

These impurities can decrease the activity of the enzymes involved in saccharification, affect the processes for purifying the sugar solutions obtained, inhibit the growth of microorganisms, and interfere with fermentation processes and purification of the compounds produced through the fermentation process.

In order to facilitate the saccharification reaction of cellulosic biomass from hygiene products, for example, the super-absorbent polymer (referred to as SAP) present must be removed or suitably treated to decrease its absorbent power. The presence of a super-absorbent polymer can, in fact, lead to a very viscous suspension or mixture during pre-treatment and/or subsequent enzyme saccharification, which is difficult to mix and transfer. It may also affect the catalytic activity of the enzymes used in the saccharification process and/or the microorganisms involved in any downstream fermentation process, if there is one. However, separation of the superabsorbent polymer from the cellulosic fraction is difficult and often not complete.

The presence of organic and/or inorganic residues can also make both the enzyme saccharification of waste cellulosic biomass and subsequent use of the sugars obtained in fermentation processes difficult, as these residues interfere with enzyme-catalysed reactions and microorganism metabolism by inhibiting their growth and the fermentation processes.

For example, patent JP5875922B2 describes a method for obtaining sugars from disposable nappies in which calcium chloride is added to a sugar solution obtained by enzyme saccharification of biomass in order to remove the super-absorbent polymer. As calcium chloride is added during or after saccharification, the super-absorbent polymer is present in the saccharification reactor and, in addition to decreasing reactor capacity by absorbing water and increasing reactor volume, interferes with enzyme activity and affects saccharification efficiency. Furthermore, the use of high concentrations of salts can cause inactivation of the enzymes during saccharification or compromise the viability of the microorganisms used for fermentation.

The present invention makes it possible to overcome the problems described above. In fact, it has been found that by subjecting waste cellulosic biomass to a particular alkaline pre-treatment it is possible to reduce the presence of impurities and obtain C5-C6 sugars suitable for use in fermentation processes. The process according to the invention in fact not only makes it possible to destructure the cellulosic fraction to make it more easily able to be attacked by enzymes, but also to remove impurities that inhibit metabolism of the microorganisms used in fermentation processes.

The object of the present invention is therefore to provide a process for producing C5-C6 sugars from waste cellulosic biomass containing impurities, comprising the steps of:
(a) contacting said biomass with a basic aqueous solution having a pH > 12, preferably ≥ 13 at a temperature of between 60 and 120° C., resulting in a mixture containing at least 5% by weight of said cellulosic biomass in relation to the total weight of the solution;
(b) separating said mixture into a solid fraction comprising cellulose and a liquid fraction;
(c) subjecting the solid fraction to one or more washes with water;
(d) subjecting the solid fraction resulting from step (c) to a hydrolysis treatment resulting in a hydrolysate comprising C5-C6 sugars.

Preferably, after step d) the process comprises a step e) of separating a liquid fraction containing said C5-C6 sugars from said hydrolysate.

In the meaning of the present invention waste cellulosic biomass means the organic fraction of plant origin, mainly comprising cellulose, (known as the cellulosic fraction) separated from post-industrial and/or post-consumer waste. Where the cellulosic fraction is derived from post-consumer biomass it preferably undergoes a sanitisation process to eliminate any pathogens present before being fed to the present process.

According to a preferred aspect, the waste cellulosic biomass according to the present invention is derived from post-consumer biomass and originates, for example, from waste sorting plants or sewage treatment plants.

According to one aspect, waste cellulosic biomass is derived from a hygiene product and may include a super-absorbent polymer.

According to one aspect, waste cellulosic biomass originates from sewage or wastewater treatment plants.

The waste cellulosic biomass comprises from 20%, preferably from 40%, to 99% by weight of cellulose with respect to the dry weight of the biomass. The cellulose content is preferably over 50%, more preferably over 55% and even more preferably 60% by weight or more.

Cellulose in waste cellulosic biomass is typically present in the form of fluff and has a molecular weight, structure and degree of polymerisation that distinguishes it from cellulose used in other products (e.g. paper). These characteristics may have changed as a result of treatments to which the waste cellulosic biomass may have been subjected (e.g. separation of other components, sterilisation, etc.).

The waste cellulosic biomass may comprise from 0% to 30%, preferably from 0% to 20%, even more preferably from 0% to 10% by weight of hemicellulose with respect to the dry weight of the biomass.

The waste cellulosic biomass may comprise lignin, in an amount not exceeding 15%, preferably not exceeding 10%, even more preferably not exceeding 5% by weight relative to the dry weight of the biomass. Advantageously, the lignin content is less than 2% by weight relative to the dry weight of the biomass. According to one aspect, the waste cellulosic biomass does not comprise lignin.

By dry weight of the biomass (also called dry matter or dry residue) is meant the weight of the residual portion of biomass after removal of the water it contains; it can be determined for example according to ASTM E1756 - 08.

The waste cellulosic biomass according to the present invention contains impurities.

By impurities, in the meaning of the present invention, are meant the components of the waste cellulosic biomass other than polysaccharides (i.e. cellulose and hemicellulose) and are less than or equal to 50% by weight, for example 1 to 50% by weight, with respect to the dry weight of the biomass. Preferably such impurities are less than or equal to 40%, more preferably less than or equal to 30%, more preferably less than or equal to 20% and even more preferably less than or equal to 10% by weight relative to the dry weight of the biomass.

The of polysaccharides content is determined, for example, using the method developed by the Laboratory for Analytical Procedures (LAP) of the National Renewable Energy Laboratory (Sluiter, A.; Ruiz, R.; Scarlata, C.; Sluiter, J.; Templeton, D.; Crocker, D: “Determination of Structural Carbohydrates and Lignin in Biomass.” Technical Report NREL/TP-510-42618, 2012), modified using a heated oil bath instead of an autoclave (provided for in section 10.1.8). The monosaccharides obtained are identified using an ion chromatograph with an amperometric detector.

The impurities content can therefore be determined by subtracting the polysaccharide content from the dry weight of the biomass.

Impurities can also be quantified, for example, by a two-step extraction process to remove water- and ethanol-soluble compounds, using the protocol developed by the Laboratory for Analytical Procedures (LAP) of the National Renewable Energy Laboratory (Sluiter, A.; Ruiz, R.; Scarlata, C.; Sluiter, J.; Templeton, D.: “Determination of Extractives in Biomass.” Technical Report NREL/TP-510-42619, 2005), using either the automated extraction procedure or via Soxhlet.

The components of the waste cellulosic biomass other than polysaccharides, such as SAP and/or organic contaminants and/or inorganic contaminants, can make it difficult to obtain fermentable C5-C6 sugars, decreasing the activity of the enzymes involved in saccharification, affecting the processes for purifying the sugar solutions obtained, and interfering with the metabolism of the microorganisms (e.g. by inhibiting their growth and/or fermentation processes) and the processes for purifying the compounds produced.

In one aspect, waste cellulosic biomass contains impurities comprising a super-absorbent polymer. When present, the super-absorbent polymer content is for example 1 to 35% by weight relative to the dry weight of the biomass. Preferably the super-absorbent polymer content is less than or equal to 35%, more preferably less than or equal to 30%, more preferably less than or equal to 20% and even more preferably less than or equal to 6% by weight relative to the dry weight of the biomass.

In the meaning of the present invention, a superabsorbent polymer means a cross-linked polymer capable of absorbing 400-1000 times its weight in water and retaining it even when subjected to pressure. Superabsorbent polymers may be made of synthetic monomers (e.g. acrylic acid, acrylamide, methacrylic acid, etc.), natural monomers (e.g. polypeptides and polysaccharides) or a combination thereof. To date, most SAPs used are of synthetic origin and the most often used monomers are acrylates or acrylamides. Among the super-absorbent polymers, polyacrylate is one of the most commonly used in the production of hygiene products. The super-absorbent polymer content can be determined by measuring the amount of water it is able to absorb.

Waste cellulosic biomass may contain impurities comprising organic contaminants. When present, organic contaminants may be from 0.1% to 40% by weight, preferably from 0.1% to 30% by weight, even more preferably from 0.1 to 20% by weight, relative to the dry weight of the cellulosic biomass. Examples of organic contaminants are organic acids, biologically active molecules used in detergents and cosmetics, proteins, fatty acids, pharmaceuticals and their derivatives, nitrogen compounds, etc.

Waste cellulosic biomass may contain impurities including inorganic contaminants. When present, the inorganic contaminants may be 0.1 to 40% by weight, preferably 0.1 to 30% by weight, even more preferably 0.1 to 20% by weight, relative to the dry weight of the cellulosic biomass. Inorganic contaminants of the waste cellulosic biomass may include one or more inorganic salts and metals such as iron, manganese, phosphorus, zinc, aluminium, chromium, nickel, lead, antimony, cadmium, copper.

According to one aspect of the invention, the starting waste cellulosic biomass contains impurities comprising at least 0.35% by weight, e.g. from 0.35% to 3.5% by weight of total nitrogen relative to the dry weight of the cellulosic biomass, and/or phosphorus in amounts of 500 mg/Kg or above, preferably of 750 mg/Kg or above and more preferably of 1000 mg/Kg or above with respect to the dry weight of the cellulosic biomass.

The process according to the invention may comprise a subsequent optional step of purifying and/or concentrating the C5-C6 sugars obtained from step e) by techniques known to those skilled in the art. Preferably, said step comprises one or more operations chosen from adsorption, dialysis, reverse osmosis, crystallisation, chromatography, evaporation, or distillation.

According to one preferred aspect, the C5-C6 sugars obtained from step e) are concentrated. The C5-C6 sugars obtained by this process are particularly suitable for use as carbon sources in fermentation processes for the production of chemical intermediates and polyhydroxyalkanoates, and require simplified operations for separating and purifying the products after fermentation.

The process according to the invention therefore comprises an optional step of growing a microbial strain capable of producing chemical intermediates and/or polyhydroxyalkanoates in the presence of a carbon source comprising the C5-C6 sugars hydrolysed in step d). This growth step is preferably preceded by separation step e) and optionally by the purification and/or concentration step described above.

These chemical intermediates are advantageously selected from: diols (preferably 1,4-butanediol), mono-alcohols, hydroxy acids, diacids, amino acids and diamines.

According to a preferred embodiment, the process according to the invention comprises an optional step of growing a microbial strain capable of producing 1,4-butanediol in the presence of a carbon source comprising, or advantageously comprising, the C5-C6 sugars hydrolysed in step d). This growth step is preferably preceded by separation step e) and optionally by the purification and/or concentration step described above.

According to an alternative embodiment, the process according to the invention comprises an optional step of growing a microbial strain capable of producing polyhydroxyalkanoates in the presence of a carbon source comprising the C5-C6 sugars hydrolysed in step d). Said growth step may be preceded by separation step e) and optionally by the purification and/or concentration step described above.

According to an alternative embodiment, the process according to the invention comprises an optional step of growing a microbial strain capable of producing diacids in the presence of a carbon source comprising the C5-C6 sugars hydrolysed in step d). This growth step is preferably preceded by separation step e) and optionally by the purification and/or concentration step described above.

The present invention therefore relates to a process for obtaining chemical intermediates and/or polyhydroxyalkanoates from waste cellulosic biomass containing impurities, comprising the steps of:
(a) contacting said biomass with a basic aqueous solution having a pH > 12, preferably ≥ 13 at a temperature of between 60 and 120° C., resulting in a mixture containing at least 5% by dry weight of said cellulosic biomass in relation to the total weight of the solution;
(b) separating said mixture into a solid fraction comprising cellulose and a liquid fraction;
(c) subjecting the solid fraction to one or more washes with water;
(d) subjecting the solid fraction resulting from step (c) to a hydrolysis treatment resulting in a hydrolysate comprising C5-C6 sugars;
(e) preferably separating a liquid fraction containing said C5-C6 sugars from said hydrolysate;
(f) optionally purifying and/or concentrating said C5-C6 sugars by one or more of the following operations: adsorption, dialysis, reverse osmosis, crystallisation, chromatography, evaporation, or distillation;
(g) growing a microbial strain capable of producing chemical intermediates and/or polyhydroxyalkanoates in the presence of a carbon source consisting of said C5-C6 sugars.

The process according to the invention may be preceded by mechanical comminution treatment of the waste cellulosic biomass prior to step a). Preferably, the biomass is reduced to a size of less than 2 cm, preferably less than 1 cm, for example by mechanical treatments such as grinding, cutting, crushing, shredding or combinations thereof. The treatment may be carried out through the use of a mill, or any means capable of reducing the size of such biomass.

The process according to the invention will now be described in more detail.

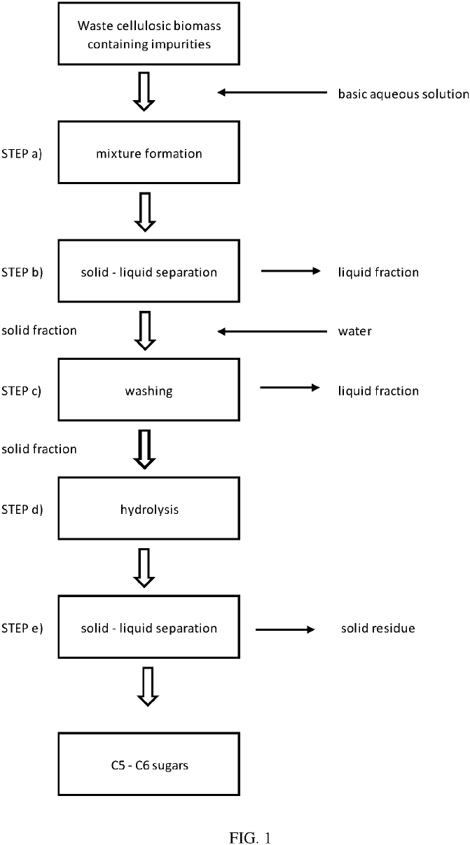

FIG. 1 shows a flow chart of the process according to the invention.

In step a) of the process, the waste cellulosic biomass containing impurities is placed in contact with a basic aqueous solution having a pH > 12, preferably ≥ 13, more preferably ≥ 13.3, resulting in a mixture containing at least 5%, preferably at least 7.5%, even more preferably at least 10% by weight of said cellulosic biomass with respect to the total dry weight of the biomass.

The basic pH of the aqueous solution can be achieved by the addition of bases such as NaOH, LiOH, KOH, Mg(OH)₂, Ca(OH)₂, alkali carbonates (e.g. Na₂CO₃, Li₂CO₃, K₂CO₃) and mixtures thereof. The use of NaOH and K₂CO₃ is preferred. The use of NaOH is particularly preferred. The base is added in amounts of less than 20%, preferably less than 15%, even more preferably less than 10% relative to the dry weight of the biomass. The cellulosic biomass is placed in contact with said basic aqueous solution at a temperature between 60 and 120° C., preferably between 70 and 100° C., even more preferably between 80 and 100° C. and preferably at atmospheric pressure. When operating at high temperatures (for example at temperatures ≥ 100° C.), it is advantageous to operate at pressures above atmospheric pressure.

The cellulosic biomass is placed in contact with said basic aqueous solution for between 30 minutes and 24 hours, preferably between 1 and 10 hours, even more preferably between 2 and 5 hours.

Before or after step a) it is possible to put the cellulosic biomass in contact with an oxidising agent. The use of such an oxidising agent makes it possible to reduce the content of any organic contaminants, such as pharmaceuticals, present in the biomass.

In a preferred embodiment the oxidising agent is hydrogen peroxide, at a concentration of said oxidising agent between 0% and 3% by weight relative to the weight of water used in step a). Step a) is preferably carried out under conditions of gentle stirring or vigorous stirring to obtain a mixture of homogeneous composition.

At the end of step a), after appropriate cooling of the mixture, the pH can be reduced by the addition of an acid, e.g. H₂SO₄, until pH values below 13, preferably below 8, are obtained. The mixture obtained in step a) is then subjected to separation into a solid fraction comprising cellulose and a liquid fraction in step b).

Such separation comprises one or more operations selected from pressing, decanting, sedimenting, centrifuging, filtering, and any other suitable technique for the separation of solids and liquids, and combinations thereof.

Preferably the mixture is passed to a device in which it undergoes a process of compression and separation into a solid fraction comprising cellulose and a liquid fraction (step b).

The material fed to step b) must nevertheless contain a quantity of solids of at least 5% or above, preferably of at least 7.5% or above, even more preferably of at least 10% or above by weight. The device for separating a solid fraction and a liquid fraction by compression which may be used in step b) may be a decanter, a settler, a filter press, a belt filter, a centrifuge, a strainer or any system commonly used for the solid-liquid separation of fibrous materials.

In a preferred embodiment of the process according to the invention, the mixture is first centrifuged or filtered using a belt filter, with the initial separation of a solid and liquid fraction. The liquid fraction obtained can be treated again, for example by filtration (e.g. microfiltration), to recover a further solid fraction rich in cellulose and hemicellulose.

Separation step b) produces a solid fraction containing mainly cellulose and hemicellulose and a liquid fraction.

The solid fraction obtained at the end of step b) has a water content that is advantageously below 60% by weight.

The solid fraction at the end of step b) is subjected to one or more washes with water or a slightly acidic aqueous solution in step c). Preferably, the washing is carried out with water.

Washing consists of adding water to the solid fraction and subsequently again separating a solid and a liquid fraction.

Washing may be performed with water and/or acidic water (pH below 7, preferably below 6) at a temperature of between 10 and 100° C., preferably between 20 and 90° C., even more preferably between 40 and 60° C., keeping the solid fraction stirred.

Washing may advantageously be carried out countercurrently.

Through the washing step the pH of the solid fraction is reduced to values below 13, preferably below 10, more preferably below 8. Those skilled in the art will be able to estimate the amount of water required to achieve this reduction in pH. Alternatively, washing may be performed until the conductivity value of the liquid fraction leaving washing is comparable to that of the water used to perform the wash.

For example, 30 to 100 ml of water may be used for each gram of dry solid fraction.

In one advantageous aspect, at the end of the washing process and between washing processes if there are two or more washes, the solid fraction is separated from the liquid fraction using the same device as in step b).

The total number of washes, the duration of each wash and the volumes of water used per wash are not particularly limiting.

Advantageously, the impurities and total nitrogen content of the solid fraction is reduced through steps a), b) and c) of the process according to the invention.

The solid fraction obtained at the end of step c) is rich in polysaccharides (i.e. cellulose and hemicellulose) and has an impurity content (for example SAP and/or organic and/or inorganic contaminants) of less than or equal to 30% by weight, preferably less than or equal to 25% by weight, more preferably less than or equal to 15% by weight and even more preferably less than or equal to 10% by weight, relative to the dry weight of the solid fraction.

Advantageously, through the present process the total ash content of the solid fraction obtained at the end of step c) is at least 50% lower than the content in the initial waste biomass. In particular, the content of aluminum, antimony, iron, manganese, molybdenum, lead and copper can be advantageously reduced by 30% or more. Among these elements, some such as aluminum, antimony and lead, typically present in cellulosic biomass waste from hygiene products or from wastewater treatment plants, are undesirable both for the reaction of enzymatic hydrolysis and for the fermentation processes with living organisms, therefore their removal makes the process of the invention particularly useful for the production of fermentable sugars. According to one aspect of the invention, starting from a waste cellulosic biomass containing impurities comprising at least 0.35% by weight, e.g. from 0.35% to 3.5% by weight of total nitrogen relative to the dry weight of the cellulosic biomass, the present process advantageously makes it possible to obtain a solid fraction at the end of step c) having a total nitrogen content of less than 0.35% by weight, preferably less than or equal to 0.2% by weight, even more preferably less than or equal to 0.1% by weight, relative to the dry weight of the solid fraction. The total nitrogen content of the solid fraction at the end of step c) is advantageously reduced of 40% by weight or more, of 50% by weight or more, preferably of 70% by weight or more and even more preferably of 80% by weight or more. The total nitrogen content may be determined, for example, using standard EN 15407:2011.

According to another aspect, when starting from a waste cellulosic biomass containing impurities comprising ≥1000 mg/Kg of phosphorus, relative to the dry weight of the cellulosic biomass, the phosphorus content of the solid fraction at the end of step c) is advantageously of ≤ 500 mg/Kg, with respect to its dry weight.

The process therefore has the further advantage of allowing the removal and possible recovery, from waste biomass, of elements such as nitrogen and phosphorus that derive from anthropogenic activities.

This solid fraction may optionally be subjected to subsequent chemical/physical or biological treatment, for example to separate the hemicellulosic and cellulosic components.

The solid fraction obtained at the end of step c) is subjected to a saccharification treatment to obtain simple sugars C5-C6 in step d) of the process. This treatment may be of the enzyme, chemical or physical type or a combination of these.

In the process according to the invention, enzyme treatment is preferred and is performed using hydrolytic enzymes or mixtures thereof capable of breaking down polysaccharides into monosaccharides.

Enzyme hydrolysis step d) may advantageously be performed by feeding a solution containing said enzymes and the solid fraction to a reactor equipped with agitation, with a concentration of the solid fraction between 5% and 30%, preferably between 10% and 25% by weight. Saccharification may be carried out by a continuous process or, alternatively, by mixing the solid fraction with said enzymes in a batch reactor.

The saccharification conditions (reaction medium, pH, temperature, duration, etc.) depend on the enzyme mixture used, in particular the presence of cellulases and hemicellulases. The addition of a buffer (based e.g. on phosphate salts) is usually requested to keep an optimal pH. In the process of the invention from waste cellulosic biomass, the enzymatic hydrolysis step d) can be unexpectedly carried out keeping the pH value constant by means of the mere controlled addition of acid / base in the reaction medium, without the need to add any buffer.

Consequently, the costs of both the reaction and the disposal of any associated waste are reduced. Furthermore, the possibility to operate without the addition of salts helps to keep low the conductivity and reduces the impact on the fermentation process and downstream.

The cellulases and hemicellulases used in the present invention may be any enzyme having a cellulase activity or a hemicellulase activity, respectively. The cellulases and hemicellulases may be part of an enzyme cocktail comprising one or more cellulases, one or more hemicellulases or a mixture thereof. Suitable enzyme cocktails are commercially available, such as CTec2 and HTec2 (Novozymes), Viscamyl Flow (Genencor, DuPont) and Cellulase 8000L (Enzyme Supplies).

Enzyme treatment can be performed in the presence of one or more bacteriostatic and/or bactericidal agents capable of counteracting the unwanted growth of microorganisms that deplete the sugar content (e.g. antibiotics, short-chain fatty acids such as nonanoic acid, parabens, etc.).

Hydrolysis may also be achieved chemically and/or physically, e.g. using mineral acids, such as HC1 and H₂SO₄, or solid acids, such as sulfonated organic resins. For example, hydrolysis may be performed using carbon catalysts in which the active species is based on sulfonic groups, such as activated sulfonated carbon, and with carbon-silica nanocomposite materials. Such solid acids are advantageously presented in macro- or mesoporous form.

A hydrolysate is obtained at the end of step d), and this preferably undergoes a step e) of separating a solid fraction and a liquid fraction containing the C5-C6 sugars (referred to as sugar solution).

Separation may be performed by exploiting the different characteristics of the solid and liquid phases (e.g. density and size of particles present), and comprises one or more of the following operations: pressing, decantation, sedimentation, centrifugation, filtration, and any other suitable technique for solid-liquid separation and combinations thereof.

The choice of the type of equipment, its combinations and its mode of operation depends on the quantity, the type of hydrolysate to be separated and the desired quality.

Separation operations may, for example, be performed by exploiting the different densities of the solid and liquid fractions, using a centrifuge or a decanter or sedimenter.

In a preferred embodiment of the process according to the invention, the C5-C6 sugars obtained from step d) are separated by at least one filtration operation, preferably ultrafiltration. Filtration operations include microfiltration and/or ultrafiltration and/or nanofiltration. According to one aspect, the C5-C6 sugars obtained from step d) are separated by centrifuging, microfiltration and ultrafiltration.

According to an alternative aspect, the C5-C6 sugars obtained from step d) are separated by microfiltration and ultrafiltration.

According to a further alternative aspect, the C5-C6 sugars obtained from step d) are separated by centrifuging or decanting and ultrafiltration.

Ultrafiltration may optionally be followed by one or more diafiltration operations.

Ultrafiltration may optionally be followed by one or more nanofiltration operations.

Microfiltration may for example be performed using 0.1 µm polysulfone membranes.

Nanofiltration may for example be performed using membranes made of polyamide with pores of 250-300 KDa.

Any ultrafiltration technique using any filter unit equipped with semi-permeable membranes, e.g. tubular, hollow-fibre, spiral, plate-and-frame type and working using a flow tangential or perpendicular to the surface of the membrane, may be used for ultrafiltration. With regard to filter membranes, semipermeable membranes made of cellulose acetate, cellulose acetate derivatives such as cellulose acetobutyrate, and synthetic polymers, such as polypropylene, polyamides, polyimides, PVDF (polyvinylidene fluoride), PAN (polyacrylonitrile), PES (polyethersulfone) and ceramics may be used. Preferably, semi-permeable polyethersulfone membranes with a porosity of 10 KDa or less are used.

The choice of temperature, transmembrane pressure and other operating conditions under which the ultrafiltration phase is performed will mainly be determined by the viscosity of the aqueous mixture fed and the type and porosity of the membrane used.

As ultrafiltration proceeds, the viscosity of the aqueous mixture and the transmembrane pressure naturally tend to increase and separation efficiency tends to decrease. This necessitates the use of gradually increasing pressures which, if too high, can damage the filter unit and impair the efficiency of the process. In order to avoid the use of excessively high pressures it is possible to resort to so-called diafiltration, feeding one or more aliquots of a restorative solution that compensates for the portion of the aqueous mixture that has permeated through the membrane. Diafiltration may be performed either continuously or discontinuously.

The process according to the invention may comprise a subsequent optional step of purifying and/or concentrating the C5-C6 sugars obtained from step e) by one or more operations chosen from adsorption, dialysis, reverse osmosis, crystallisation, chromatography, evaporation, or distillation.

The choice of the type of equipment, the combinations thereof and their mode of operation will depend on the quantity and type of hydrolysate to be purified and/or concentrated, and the desired quality.

The liquid fraction obtained at the end of step e) may optionally be concentrated to decrease operating volumes during the subsequent fermentation process.

Concentration may be performed by known techniques, for example by distillation, evaporation or reverse osmosis, until a syrup with a C5-C6 sugars concentration of between 20% and 80% by weight, preferably between 40% and 80% by weight, is obtained. Operations that do not require excessively high temperatures are preferred, to avoid the formation of degradation byproducts that may have an inhibiting effect on the microorganisms used in fermentation.

The sugar solution comprising C5-C6 sugars obtained after step e) of the process according to the invention has an impurity content of less than 45% by weight, preferably less than 35% by weight, more preferably less than 25% by weight, even more preferably less than 15% by weight, with respect to the dry weight of said sugar solution, which makes it particularly suitable for use in fermentation processes. This impurities content does not in fact interfere with microorganism metabolism.

The impurities content in the sugar solution is calculated by subtracting the sugar content from the dry weight of the sugar solution.

In a second aspect, the present invention therefore relates to a composition of C5-C6 sugars obtained from waste cellulosic biomass having an impurity content of less than 45% by weight preferably of less than 35% by weight, more preferably less than 25% by weight, even more preferably less than 15% by weight, relative to the dry weight of the composition.

According to an aspect of the invention, said composition of C5-C6 sugars has a total nitrogen content of from 0.0% by weight to 0.5% by weight, preferably from 0.1% by weight to 0.5% by weight, more preferably from 0.3% by weight to 0.5% by weight relative to the dry weight of the C5-C6 sugar composition.

According to another aspect of the invention, said composition of C5-C6 sugars has a phosphorus content of from 0.0% by weight to 2.5% by weight, preferably from 0.25% by weight to 2.5%, by weight more preferably from 1.25% by weight to 2.5% by weight relative to the dry weight of the C5-C6 sugar composition.

According to a preferred aspect, said composition of C5-C6 sugars has a total nitrogen content of from 0.0% by weight to 0.5% by weight and a phosphorus content of from 0.0% by weight to 2.5% by weight, relative to the dry weight of the C5-C6 sugar composition.

The C5-C6 sugars of said composition can therefore be biochemically transformed (e.g. fermentation by bacteria, archaea or yeasts) to obtain polyhydroxyalkanoates and chemical intermediates such as, for example, diols (preferably 1,4-butanediol), mono-alcohols, hydroxy acids, diacids and amino acids.

Said polyhydroxyalkanoates (PHA) are preferably selected from the group consisting of: polyhydroxybutyrate, polyhydroxybutyrate-valerate, polyhydroxybutyrate-propanoate, polyhydroxybutyrate-hexanoate, polyhydroxybutyrate-decanoate, polyhydroxybutyrate-dodecanoate, polyhydroxybutyrate-hexadecanoate, polyhydroxybutyrate-octadecanoate, poly 3-hydroxybutyrate-4-hydroxybutyrate. More preferably said polyhydroxyalkanoates are selected from the group consisting of polyhydroxybutyrate (PHB), polyhydroxybutyrate-valerate (PHBV) and polyhydroxybutyrate-hexanoate (PHBH).

These chemical intermediates are preferably selected from the group consisting of: diols such as 1,2-ethanediol, 1,2-propanediol, 1,3-propanediol, 1,3-butanediol, 2,3-butanediol, 1,4-butanediol, mono-alcohols such as butanol and ethanol, hydroxy acids such as lactic acid, diacids (DCA), such as succinic, glutaric, adipic, muconic, azelaic, sebacic, undecanedioic, dodecanedioic, brassylic, hexadecanedioic, octadecanedioic, octadecenedioic, octadecadienoic, octadecatrienoic, eicosanedioic, docosanedioic and furandicarboxylic acids, amino acids such as alanine, arginine, asparagine, cysteine, glycine, glutamine, histidine, methionine, proline, tyrosine, valine, leucine, isoleucine, aspartic and glutamic acid, lysine, threonine, serine, tryptophan and phenylalanine.

Examples of biochemical transformations for the production of polyhydroxyalkanoates are fermentations carried out by bacteria belonging to the genera Bacillus, Rhodococcus, Pseudomonas, Ralstonia, Haloferax, Cupriavidus, Protomonas, Alcaligenes, Escherichia and Leuconostoc.

In order to produce PHA the bacterial culture may first be grown in a suitable medium to promote the production of cell biomass, and then the growth conditions may be changed to induce the synthesis and accumulation of PHA in the form of intracellular inclusions. The synthesis of PHA is usually induced by subjecting the microorganism to a deficiency of macronutrients such as phosphorus, nitrogen and sulphur, and simultaneously to an excess of carbon sources.

Examples of biochemical transformation for the production of chemical intermediates are fermentation by bacteria (e.g. E. coli) or oleaginous yeasts such as those belonging to the genera Yarrowia, Candida, Rhodotorula, Rhodosporidium, Cryptococcus, Trichosporon and Lipomyces. Particularly preferred are yeasts belonging to the genera Yarrowia and Candida.

For example, mixtures of C5-C6 sugars can be used by genetically modified E. coli in the process described in patent WO 2015/158716 to obtain 1,4-butanediol (1,4-BDO).

The 1,4-BDO can be obtained by a fermentation process from a culture medium containing at least one sugar, preferably glucose and optionally one or more sugars other than glucose, in the presence of one or more microorganisms having at least one metabolic pathway for the synthesis of 1,4-BDO.

The sugars supplied to the microorganisms for the production of 1,4-BDO may be C5-C6 sugars derived from the saccharification of waste cellulosic biomass or mixtures thereof with first-generation sugars, characterised by a high level of purity. In the case of mixtures, these may comprise from 1 to 99% by weight, preferably from 15 to 65% by weight, of sugars derived from the saccharification of waste cellulosic biomass with respect to total sugars.

The culture medium may comprise other substances necessary for the growth and sustenance of the microorganisms during the fermentation phase, such as elements such as C, H, O, N, K, S, P, Fe, Ca, Co, Mn, Mg. Typically, the culture medium may comprise one or more components selected from the group consisting of sugars other than glucose, protein hydrolysates, proteins, amino acids, organic acids, vitamins, mineral salts, yeast extracts, and trace elements such as Cobalt, Calcium and Copper. Cobalt, calcium and copper can be dosed into the culture medium, for example, as salts such as Cobalt chloride, Calcium chloride and Copper chloride. Generally, the culture medium comprises at least one sugar, usually glucose and optionally one or more sugars other than glucose, in concentrations between 10 and 100 g/L. Since during the fermentation stage of the present process the microorganisms consume one or more sugars, it is generally necessary to reintroduce these sugars into a fermentation reactor. This reintroduction can be carried out in a continuous or discontinuous manner, according to methods known to those skilled in the art.

To limit the content of unused sugars and thus optimise the economy of the process, the supply of one or more sugars is advantageously interrupted or gradually decreased before the end of fermentation. As regards other components of the culture medium, the culture medium generally contains salts, essential minerals, and antifoaming agents. The culture medium may be prepared in any manner known to the those skilled in the art, for example by mixing all components together or by pre-mixing all components excluding glucose and adding them later, either individually or already pre-mixed. It is also possible to use a commercially available culture medium as a starting point and suitably modify its composition at a later stage, for example when bringing the culture medium into contact with the microorganism having at least one metabolic pathway for the synthesis of 1,4-BDO from a renewable source. During fermentation, the combination comprising the microorganism and the culture medium comprising one or more sugars is maintained under conditions suitable for exploiting the metabolic pathway for the synthesis of 1,4-BDO from renewable sources. Furthermore, those skilled in the art will be able to check the progress of the process during fermentation, for example by checking one or more parameters and possibly acting on them to bring the process back to conditions suitable for the production of 1,4-BDO.

Mixtures of C5-C6 sugars can also be used by oleaginous yeasts belonging to the Candida genus to obtain diacids.

Diacids may be produced by means of a two-step fermentation, i.e. with a biomass cell growth step and a subsequent production step. In the initial growth step, the cells grow using the sugar present in the culture medium as their sole carbon source. The subsequent DCA production step is preferably a fed-batch process aimed at keeping the cell biomass active and catalytically active in converting fatty acids to DCA. Advantageously, this step has a dual feed: a sugar to keep the cells active and a source of monocarboxylic acids or glycerides of monocarboxylic acids for biotransformation.

The C5-C6 sugars obtained by the process according to the invention may also undergo transformation by chemical means to produce chemical intermediates. Examples of chemical transformation are the isomerisation of glucose to fructose and subsequent dehydration in an acidic environment to obtain HMF, which in turn can be oxidised to obtain furandicarboxylic acid and derivatives thereof.

The chemical intermediates that can be obtained by transformation of sugars brought about by the process according to the invention, such as butanediol, succinic acid, adipic acid, muconic acid, furandicarboxylic acid, terephthalic acid, levulinic acid, lactic acid and polyhydroxyalkanoates, are useful as monomers for the synthesis of polymers, in particular polyesters.

The process according to the invention will now be described according to a non-limiting example.

### EXAMPLES

### Example 1

### Step a)

Cellulosic biomass from adult absorbent products used for this example had a moisture content of 10.45%, impurity content of 27, 4% by weight and total nitrogen 0.56% by weight, relative to the dry weight of the biomass. The impurities content was determined by subtracting the polysaccharide content from the dry weight of the biomass according to Technical Report NREL/TP-510-42618, 2012 as reported above.

6.7 kg of such biomass were added to a cylindrical reactor equipped with a mechanical stirrer with alternating paddles, a temperature control system, pH meter and drip funnel, in a final concentration of 10% and 59.3 litres of a basic aqueous solution, resulting in a mixture with a pH of 13.3. The resulting mixture was then heated to a temperature of 90° C. by means of a heating oil jacket and maintained under gentle agitation for 4 hours.

### Step b)

The mixture obtained at the end of step a) was separated by a centrifuge filter bag, yielding 10 kg of a solid fraction including cellulose and 56 litres of a liquid fraction.

### Step c)

The solid fraction comprising cellulose from step b) was washed successively with 330 litres of water at a temperature of 20° C. until a pH of approximately 8 was reached.

At the end of step c) the solid fraction had an impurity content of 5% by weight and a total nitrogen content of 0.28% by weight, relative to the dry weight of the solid fraction.

### Step d)

The solid fraction from step c) underwent enzyme hydrolysis treatment.

3.6 kg of dry solid fraction was added to 23.3 litres of 50 mM phosphate buffer at pH 5 in a cylindrical reactor equipped with a mechanical stirrer with alternating paddles, a temperature control system and a pH control system, and 569 ml of Viscamyl™ Flow (an enzyme complex containing enzymes with cellulolytic and hemicellulolytic action) and 24 ml of nonanoic acid was added. The reaction was maintained at 50° C. under gentle agitation for 48 h.

### Step e)

On completion of the hydrolysis reaction, the hydrolysate was centrifuged, filtered through sieves with a mesh size of up to 25 micrometers and subjected to tangential ultrafiltration using regenerated cellulose membranes with 10 kDa pores, resulting in a liquid fraction (sugar solution) with a glucose concentration in solution of 55 g/L, determined by ion chromatography. At the end of step e) the liquid fraction had a C5-C6 sugar content equal to 76.66% by weight with respect to the dry weight of the liquid fraction. The content of impurities, obtained by subtracting the content of glucose, xylose, oligosaccharides and additives of step d) from the dry weight of the liquid fraction, was equal to 15.93% by weight, with respect to the dry weight of the liquid fraction.

The sugar content was analysed using a Metrohm Professional IC Vario 940 ion chromatograph, equipped with an amperometric detector and fitted with a Metrosep Carb 2 250 mm x 4.0 mm x 5 µm column and Metrosep Carb 2 Guard/4.0 pre-column, using the following operating conditions:
Flow: 0.7 mL/minute
Oven temperature: 30° C.
Detector temperature: 35° C.
Eluent: 40 mM NaOH + 40 mM NaOAc.

### Example 2

The liquid fraction obtained at the end of step e) was concentrated using a rotary evaporator under vacuum at 50° C., resulting in a syrup with a glucose concentration in solution of 484.4 g/L, determined by liquid chromatography.

The syrup obtained was used as a carbon source in a fermentation process for the production of 1,4-BDO.

A strain of Escherichia coli with a metabolic pathway for the synthesis of 1,4-BDO was inoculated into a 250 ml Erlenmeyer flask containing 50 ml of first culture medium (10 g/l of Tryptone enzymatic digest from casein Sigma, 5 g/l of Yeast extract Sigma, 0.5 g/l of NaCl, 10 g/l of glucose). The flask was then shaken at 275 rpm overnight, at a temperature of 35° C., yielding a preinoculum.

Subsequently an aliquot of the preinoculum was transferred to a 1000 ml Erlenmeyer flask containing 200 ml of a second culture medium (12.78 g/L M9 Minimal Salt Teknova; 10 g/L first-generation glucose; 1 ml/L 1 M MgSO₄; 1 ml/L 0.1 M CaCl₂; 1ml/L Trace Elements Teknova T1001; 0.5 ml/L Streptomycin sulphate salt 100 mg/ml).

The Erlenmeyer flask was incubated at 35° C., shaking the contents at 275 rpm for approximately 8 hours. After this incubation period the optical density reached an OD value (optical density measured at 600 nm) of approximately 3 to 4 OD and the culture was used to inoculate a seed fermenter.

After reaching proper cell biomass, an aliquot of the seed fermentation was used to inoculate at OD 4 a production fermenter containing 1 litre of medium (1.73 g/L KH₂PO₄; 0.83 g/L (NH₄)₂SO₄; 0.30 g/L Na₂SO₄; 0.038 g/L Ca Citrate*4H₂0; 0.20 g/L Citric Acid C₆H₈O₇; 1 M MgSO₄ (2 ml/L); Trace Elements Teknova T1001 2 ml 1/L; Antifoam 204 Sigma 0.1ml/L) and 20 g/L of first-generation glucose to promote the growth of the microorganism before the addition of sugar solution from Example 1.

The sugar from Example 1, purified and concentrated, was progressively fed into the fermenter in a fed-batch process so as to keep the glucose concentration in the culture medium constant in the range 30-60 g/L, and then gradually reduced until a glucose concentration at the end of fermentation (about 30 - 40 hours after inoculation) of about 0 g/L was obtained.

The bioreactor was maintained under stirring >700 rpm and air flow at 0, 6755 Pa*m3/s, and optimized pH and temperature conditions.

Samples of the reaction medium were taken at different times to assess the production of 1,4-BDO by analysis using ion chromatography.

The 1,4-BDO content was determined using a Metrohm Professional IC Vario 940 ion chromatograph, equipped with amperometric detector and fitted with a Metrosep Carb 2 250 mm x 4.0 mm x 5 µm column and a Metrosep Carb 2 Guard/4.0 pre-column, using the following operating conditions:
Flow: 0.7 mL/minute
Oven temperature: 30° C.
Detector temperature: 35° C.
Eluent: 50 mM NaOH + 5 mM NaOAc.

Based on the data collected, Titre and Productivity were determined (Table 1), where:
“Titre” (g/1): weighted concentration of 1,4-BDO in the reaction medium at the end of the fermentation time;
“Productivity” (g/1/h): weighted average synthesis rate of 1.4-BDO, calculated as

### Titre/Hours of Fermentation

The results obtained are shown in Table 1.

### Example 3

The same fermentation process described in Example 2 was performed using a concentrated syrup prepared by mixing 25% by weight of glucose from Example 1 as a carbon source, and 75% by weight of first-generation glucose.

Fermentation was complete approximately 40 hours after inoculation.

The results obtained are shown in Table 1.

### Comparative Example 4

665.7 g of cellulosic biomass from adult absorbent products (with a moisture content of 9.59%, impurity content of 27, 4% by weight and nitrogen content of 0.56% by weight, relative to the dry weight of the biomass) was subjected directly to an enzyme hydrolysis without any additional treatment.

Cellulosic biomass from adult absorbent products was introduced into a cylindrical reactor equipped with a mechanical stirrer with alternating paddles, a temperature control system and a pH control system, in the presence of 11.87 L of 50 mM phosphate buffer at pH 5, 95.1 ml of Viscamyl™ Flow (enzyme complex containing enzymes with cellulolytic and hemicellulolytic action) and 12 ml of nonanoic acid. The reaction was maintained at 50° C. with gentle agitation for 140 hours.

On completion of the hydrolysis reaction the hydrolysate was centrifuged, filtered through sieves with a mesh size of up to 25 micrometers and underwent tangential filtration through regenerated cellulose membranes with 10 kDa pores, resulting in a liquid fraction with a glucose concentration in solution of 29.5 g/L, determined by ion chromatography.

The liquid fraction obtained was concentrated using a rotary evaporator under vacuum at 50° C., resulting in a syrup with a concentration of glucose in solution of 467.9 g/L, determined by ion chromatography.

The same fermentation process described in Example 2 was performed using as the carbon source a mixture prepared by mixing 25% by weight of glucose produced in Comparative Example 4 and concentrated, and 75% by weight of first-generation glucose.

Fermentation was stopped about 27 hours after inoculation due to a drastic reduction in the microorganism vital parameters.

The results obtained are shown in Table 1.

TABLE 1

| Example | % sugar from cellulosic biomass fed during fermentation | Titre (g/L) | Productivity (g/L/h) |
|---|---|---|---|
| 2 | 100 | 86.26 | 2.17 |
| 3 | 25 | 119.81 | 2.67 |
| 4 comparative | 25 | 22.94 | 0.856 |

The results obtained clearly show that the process according to the invention can be used to obtain C5-C6 sugars from a waste cellulosic biomass suitable for use by a microbial strain capable of producing 1,4-butanediol. Such sugars, used alone (Example 2) or in a mixture with first-generation sugars (Example 3), do not interfere with the cell viability and are efficiently converted by it into 1,4-butanediol, as demonstrated by the titre and productivity values shown in Table 1.

On the other hand Comparative Example 4 demonstrates that sugars obtained from a waste cellulosic biomass which have not undergone the process according to the invention cannot be used in fermentation, even when mixed with a first-generation sugar. Indeed, such sugars have an impurity content that makes them strongly toxic for thecell viability. Indeed, the presence of the impurities caused a drastic reduction in its vital parameters and fermentation was therefore stopped only 28 hours after inoculation.

In addition, the presence of impurities interfered with the production of 1,4-butanediol, causing lowering of the fermentation parameters.

### Example 5

### Step a)

0.96 Kg of upcycled cellulose biomass from a sewage treatment plant with a moisture content of 6.69% (impurity content of 10.92% and total nitrogen content of 1.14% relative to the dry weight of the biomass) was diluted in a stirred jacketed reactor at a final concentration of 5.5% wt/wt in 15.35 liters of basic solution with a final pH of 13. The resulting mixture was heated at 90° C. and gently stirred for 4 h. At the end of the process the mixture was cooled down and 1.25 Kg of an aqueous solution of H₂SO₄ 7% wt was added up to the neutralization of the solution.

### Step b)

The mixture obtained at the endo of step a) was filtered using a filterbag-centrifuge obtaining 2.6 Kg of a solid fraction including cellulose and 14.9 litres of a liquid fraction.

### Step c)

The solid fraction containing cellulose from step b) was washed with 47.1 L of water at 20° C. At the end of step c) the solid fraction showed an impurity content of 5.45% by weight (as above measured as the sum of water extractives and ethanol extractives) and a total nitrogen content of 0.33% by weight, relative to the dry weight.

### Step d)

The washed solid fraction from step c) underwent an enzymatic hydrolysis treatment. 0.617 Kg of the solid fraction was diluted with 5.57 L of deionized H₂O inside a stirred tank bioreactor equipped with mechanical stirrer, thermal jacket to control temperature and pH control system. pH was set to 5 and automatically corrected using H2SO4 0.3 M and NaOH 0.6 M. In this case the reaction was performed without addition of further salts, advantageously obtaining a final sugar solution with reduced conductivity and thus a reduced impact on the fermentation and downstream process. 5.7 mL of nonanoic acid 97% wt and 90 mL of Genencor Viscamyl™ Flow were added to the reaction mixture. The reaction was maintained to 50° C. and gently stirred for about 90 h until no further increment in the concentration of glucose in the solution can be observed

### Step e)

On completion of the hydrolysis reaction, the hydrolysate was decanted to separate the liquid fraction containing sugars by the not-digested solid fraction. The liquid fraction was filtered in tangential flow microfiltration using 0.1 µm membrane and tangential flow ultrafiltration using 5 KDa polyethersulfone (PES) membrane. Retentate was subjected to diafiltration to maximize sugar recovery.

The obtained liquid fraction had a glucose concentration of 29.5 g/L and xylose concentration of 3.5 g/L.

Analysis to quantify sugar concentrations were performed with high pressure liquid chromatography (HPLC) using a HPLC Surveyor Thermo Scientific, equipped with Refractive Index Detector (RID) Shodex and fitted with a Phenomenex Rezex ROA-Organic Acid H+ 300 x 7.8 mm column and a Phenomenex Carbo-H 4 x 3.0 mm ID pre-column, using the following operating conditions:
Flow: 0.6 mL/minute, isocratic
Oven temperature: 65° C.
Eluent: 5 mM Sulfuric Acid

The operations of steps a) to c) have therefore led to an enrichment in cellulose of the upcycled cellulose biomass, and a consequent greater release of glucose. Additionally, they allowed to slightly increase the yield of hydrolysis with respect to the same hydrolysis reaction performed directly on the same starting biomass.

### Example 6

Final purified sugar solution deriving from example 5 step e was concentrated using rotary evaporator working in vacuum at 50° C. The syrup obtained had a glucose concentration of 526 g/L and xylose concentration of 62 g/L.

The syrup was mixed with 1st generation glucose reaching a glucose final ratio of 30% wt in the mixture (30% glucose from Example 5 and 70% 1st generation glucose). The mixture obtained was used as a carbon source to feed a fermentation process for 1,4-bioBDO production according to example 2 with minor modifications.

The chromatograpy analysis showed the production of 1,4-BDO with titer of 117 g/L at the end of the fermentation time (about 35 h) and a productivity of 3.38 g/L/h was observed.

1. A process for the production of C5-C6 sugars from waste cellulosic biomass containing nitrogen, comprising the steps of:
(a) placing said biomass in contact with a basic aqueous solution of pH > 12 at a temperature between 60 and 120° C., obtaining a mixture containing at least 5% by dry weight of said cellulosic biomass in relation to total weight of the solution;
(b) separating said mixture into a solid fraction comprising cellulose and a liquid fraction;
(c) subjecting said solid fraction to one or more washes with water;
(d) subjecting the solid fraction resulting from step c) to a hydrolysis treatment resulting in a hydrolysate comprising C5-C6 sugars,
in which the waste cellulosic biomass is a post-consumer biomass and/or a post-industrial biomass.

2. The process according to claim 1, in which the waste cellulosic biomass is derived from a hygiene product.

3. The process according to claim 1, in which the waste cellulosic biomass comes from wastewater treatment plants.

4. The process according to claim 1, in which the waste cellulosic biomass has an impurity content of less than or equal to 50% by weight, relative to the dry weight of the biomass.

5. The process according to claim 1, in which the waste cellulosic biomass contains impurities comprising a super-absorbent polymer.

6. The process according to claim 5, in which said cellulosic biomass has a super-absorbent content of less than or equal to 35% by weight relative to the dry weight of the biomass.

7. The process according to claim 1, in which the total nitrogen content is from 0.35% to 3.5% by weigh, relative to the dry weight of the cellulosic biomass.

8. The process according to claim 1, in which the waste cellulosic biomass contains impurities comprising phosphorus.

9. The process according to claim 1, in which the solid fraction obtained at the end of step c) has an impurity content of less than or equal to 30% by weight, relative to the dry weight of the solid fraction.

10. The process according to claim 1, 9, in which the solid fraction obtained at the end of step c) has a total nitrogen content of less than 0.35% by weight, relative to the dry weight of the solid fraction.

11. The process according to claim 1, in which the solid fraction obtained at the end of step c) has a phosphorus content of less than 500 mg/Kg, relative to the dry weight of the solid fraction.

12. The process according to claim 1 comprising a subsequent step e) of separating said C5-C6 sugars from said hydrolysate.

13. The process according to claim 12 comprising a subsequent step of purifying and/or concentrating the C5-C6 sugars obtained from step e) through one or more operations selected from adsorption, dialysis, reverse osmosis, crystallisation, chromatography, evaporation, distillation.

14. The process according to claim 1 comprising a subsequent step of growing a microbial strain capable of producing chemical intermediates and/or polyhydroxyalkanoates in the presence of a carbon source comprising the C5-C6 sugars hydrolysed in step d).

15. The process according to claim 14 comprising a step of growing a microbial strain capable of producing 1,4-butanediol in the presence of a carbon source comprising the C5-C6 sugars hydrolysed in step d).

16. The process according to claim 1, in which said waste cellulosic biomass undergoes mechanical comminution treatment prior to step a).

17. The process according to claim 1, in which in step a) the biomass is placed in contact with a basic aqueous solution for a time of between 30 minutes and 24 hours.

18. A composition of C5-C6 sugars obtained from waste cellulosic biomass having an impurity content of less than 45% by weight in relation to the dry weight of the composition and a total nitrogen content of from 0.1% by weight to 0.5% by weight.

19. The process according to claim 2, in which the waste cellulosic biomass has an impurity content of less than or equal to 50% by weight, relative to the dry weight of the biomass.

20. The process according to claim 3, in which the waste cellulosic biomass has an impurity content of less than or equal to 50% by weight, relative to the dry weight of the biomass.
